(19) United States
(12) Patent Application Publication
(10) Pub. No.: US 2022/0338871 A1
(43) Pub. Date: Oct. 27, 2022

(54) METHOD AND APPARATUS FOR HOLDING SURGICAL SUTURES

(71) Applicant: Jessica Adams, Sandy, UT (US)

(72) Inventor: Jessica Adams, Sandy, UT (US)

(21) Appl. No.: 17/726,439

(22) Filed: Apr. 21, 2022

Related U.S. Application Data
(63) Continuation-in-part of application No. 17/334,047, filed on May 28, 2021.
(60) Provisional application No. 63/178,654, filed on Apr. 23, 2021.

(51) Int. Cl. A61B 17/06; A61B 90/94

(52) U.S. Cl. CPC A61B 17/06061; A61B 17/06133; A61B 90/94

(57) ABSTRACT

A method and apparatus for holding a suture needle may include a suture needle holding pad disposed on the exterior of the container holding the suture needle and suture. A method of using the container may include removing a needle/suture combination from the container and then attaching the needle to the suture needle holding pad disposed on the exterior of the container.

### RELATED APPLICATIONS

This application claims priority to and incorporates by reference U.S. Provisional Application Ser. No. 63/178,654, filed Apr. 23, 2021 and U.S. patent application Ser. No. 17/334,047, filed May 28, 2021.

### BACKGROUND

### State Of The Art

The present invention relates to a method and apparatus for holding a surgical suture.

### FIELD OF ART

Medical procedures have advanced to the point where doctors routinely conduct complex surgeries on patients for a variety of reasons. In some cases, the surgeries involve removal of cancerous or otherwise diseased tissues. In other cases, the surgery is meant to minimize the injury done during an accident. And in still other procedures surgeons are able to improve a person's appearance by various plastic surgery techniques. Regardless of the purpose of the surgery, virtually all require some suturing of the skin and/or other tissues of the patient.

Depending on the type of surgery and the types of tissues involved, a surgeon may elect to use a variety of different suture needles. Common surgical needles include ⅝ circle, ½ circle, 3/8 circle, 1/4 circle, J-shape, compound curve, 1/2 curved and straight. Each of these needles may also have a point type such as a taper point, blunt taper point, cutting edge, reverse cutting edge, taper cut and micro-point spatula. In the course of a surgery, a surgeon may elect any combination of needle shapes and points depending on the types of tissue which need to be closed and the types of incisions which were formed.

Additionally, there are numerous different suturing threads, including polypropylene monofilament, polyester, polyester braided, nylon monofilament, silk, silk braided, and many others. These materials come in different sizes and are often color coded to allow the surgeon to verify what material he or she is working with. When combined with the different needle shapes and needle points, there are literally hundreds of potential combinations which may be used.

Because of the harm which has been caused from needles left in patients during surgeries, many states and virtually all hospitals require that every surgical needle/suture be maintained in the operating field and accounted for before the patient is closed up at the end of the surgery. FIG. 1 shows a surgical tray 4 having a plurality of surgical suture packs 8a-8d disposed thereon in accordance with principles of the prior art. Suture packs 8a-8d show the suture packs prior to opening. Inside the suture packs are usually a sterilized suture tray 12a-12j which is typically made from cardboard or plastic. Suture trays 12e-12j show suture trays for which the exterior packaging has been discarded.

The suture tray 12a-12j usually includes a needle 16e-16j and a length of suture thread 20e-20j of the desired material and thickness needed for the surgery. The needle is usually held in place by a needle holder 18e-18j which is commonly formed from two plastic posts with the needle press-fit between the posts.

A portion of the thread 20e-20j is usually covered by a label 22e-22j made of paper or the like which is attached to the top of the suture tray 12e-12j. The labels 22e-22j have printed thereon information about the type of needle and the type/size of the suture material. The labels 22e-22j also typically cover at least part of the needle to protect the needle from getting damaged or catching on things, while leaving an opening 23e-23j through which the needle 16e-16j and the suture material 20e-20j can be withdrawn.

When a particular needle/suture thread is needed, the surgical technician in the operating room is responsible to grasp the desired needle with hemostats/forceps and hand it to the surgeon. When the surgeon is finished making the needed sutures, the needle is returned to the surgical technician who is responsible for securing the needle so that it can be used again as needed, and so that the surgical technician can readily identify which needle/suture combination is which.

Surgical technicians are often also required to hold retractors and other instruments to assist the surgeon. Thus, the surgical technician may be required to secure the needle with one hand, while the other hand is being used to assist the surgeon with other tools. When a surgery is complex, the surgical technician may be dealing with ten or more different needle/suture combinations, as well as other surgical tools. Thus, it is easy to lose track of exactly which needle/suture combinations is in which location.

In an attempt to control the needles, many surgical technicians have resorted to using a foam block or sponge 30 into which the suture needles are secured when not in use. To further prevent the wrong needle/suture combination being given to the surgeon, some surgical technicians write on the sponge with a marker during surgery to indicate which needle/suture combination is which. It will be appreciated that to the naked eye, it is difficult to distinguish between a size 4 silk suture and a size 6 silk suture. While the size 6 silk suture may hold an incision closed, a size 3 or 4 may be more likely to break, thereby creating potential issues after the surgery. While writing the numbers helps identify which suture is which, it is also distracting during the surgery and if the surgical technician's attention is drawn away by the surgeon, it is easy to confuse the different needle/suture combinations or to place one in the wrong place.

While a surgical technician can write a code for the various needle/suture thread combinations which are likely to be used in advance, doing so requires the surgical technician to constantly determine which needles are disposed on the sponge, which packets have not yet been opened, and which needles are in use. This can be particularly confusing if a suture breaks when the needle is pulled from the suture tray 12. If, for example, a 4 silk ½ circle needle is pulled out and the suture breaks, the surgical technician may dispose the needle in the sponge at the number indicated. He or she must then get another 4 silk ½ circle package and open it for use. While the surgeon uses the needle, the location on the sponge for the 4 silk ½ circle is already filled—potentially leading to confusion. When there are unfilled numbers on the sponge, the technician must determine if the needle is missing or has yet to be used.

Additionally, at the end of the surgery, every suture needle must be accounted for. If one is missing, the surgical technician must figure out which one is missing and why. While knowing exactly which needle/suture combination is missing may help indicate places to look, this is only possible if one knows exactly which one is missing. Thus, there is a need for an improved method and apparatus for securing surgical needles.

The complication and stress for a surgical technician can be multiplied when there are multiple surgeons or other medical professionals suturing at the same time. For example, surgeon 1 may need a certain needle/suture combination at the same time surgeon 2 needs a different needle/suture combination. If they both get returned at approximately the same time, it is easy to confuse the two and place them in the wrong positions.

### SUMMARY OF THE INVENTION

The following summary of the present invention is not intended to describe each illustrated embodiment or every possible implementation of the invention, but rather to give illustrative examples of application of principles of the invention.

A method and apparatus for holding surgical sutures may include disposing a suture needle holding pad on the suture tray or a cover thereof. The suture needle holding pad may be made from open cell or closed cell foam, styrofoam, rubber, or even a magnet or other material which will receive and hold the needle in place. In a preferred embodiment, the surgical suture holding pad is made from a material which will not damage the tip of the needle when the needle is inserted therein and will allow the needle to be pulled out so that it can be used again. Alternatively, the suture needle holding pad could be made from a magnet which holds the needle without requiring insertion of the needle tip.

In accordance with one aspect of the invention, the suture needle holding pad is disposed on the label which includes the information about the needle and suture so that the surgical technician and the surgeon can readily identify which needle/suture combination is present.

In accordance with another aspect of the invention, the suture needle holding pad is disposed on the cover while the needle holder is disposed in a different location, thereby instantly conveying to the surgical technician and the surgeon that the needle has been used if the needle is secured in the suture needle holding pad rather than in the needle holder.

In accordance with one aspect of the invention, only a single needle/suture combination is used at a time so that every needle/suture combination used in the operating field is attached to the proper tray except the one that is in use, thereby preventing the possibility of needles getting mixed up.

In accordance with another aspect of the invention, a needle/wet suture material is disposed in a fluid tight envelope having a needle holding pad attached thereto, so that once the envelope has been open and the needle used, the needle may be mounted in a needle holding pad disposed on the envelope, preferably near the location of the information regarding the needle.

### BRIEF DESCRIPTION OF THE DRAWINGS

Various embodiments of the present disclosure are shown and described in reference to the numbered drawings wherein:

FIG. 1 shows a surgical tray in accordance with the prior art;

FIG. 2 shows a top view of a surgical suture package in accordance with the principles of the present disclosure;

FIG. 3 shows a top view of the surgical suture tray of FIG. 2 with the packaging removed.

FIG. 4 shows a top view of the tray of FIG. 3 with the suture needle and suture thread removed;

FIG. 4A shows an embodiment wherein the label is disposed in the bottom of the suture tray.

FIG. 5 shows a side view of the suture tray of FIGS. 2-4;

FIG. 6 shows a top view of the surgical suture tray of FIGS. 2-5 with the suture needle attached to the surgical suture holding pad;

FIG. 7 shows a Mayo tray having a number of surgical suture trays disposed thereon;

FIG. 8 shows a method of securing a surgical suture needle on a suture tray;

FIG. 9 shows an alternate method for securing a surgical suture needle;

FIG. 10 shows a side view of a suture tray disposed inside of packaging;

FIG. 11 shows a side view of an envelope containing a needle and wet suture material;

FIG. 12 shows a top view of the envelope of FIG. 11; and

FIG. 13 shows a top view of a Mayo tray configured for use in accordance with the present disclosure.

It will be appreciated that the drawings are illustrative and not limiting of the scope of the invention which is defined by the appended claims. The embodiments shown accomplish various aspects and objects of the invention. It will be appreciated that it is not possible to clearly show each element and aspect of the present disclosure in a single figure, and as such, multiple figures are presented to separately illustrate the various details of different aspects of the invention in greater clarity. Similarly, not all configurations or embodiments described herein or covered by the appended claims will include all of the aspects of the present disclosure as discussed above.

### DETAILED DESCRIPTION

Various aspects of the invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. The drawings and the descriptions thereof are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

Reference in the specification to “one embodiment,” “one configuration,” “an embodiment,” or “a configuration” means that a particular feature, structure, or characteristic described in connection with the embodiment may be included in at least one embodiment, etc. The appearances of the phrase “in one embodiment” in various places may not necessarily limit the inclusion of a particular element of the invention to a single embodiment, rather the element may be included in other or all embodiments discussed herein.

Furthermore, the described features, structures, or characteristics of embodiments of the present disclosure may be combined in any suitable manner in one or more embodiments. In the following description, numerous specific details may be provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein, but is extended to include equivalents thereof as would be recognized by those of ordinary skill in the relevant art. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of that aspect in the claims.

It should also be noted that, as used in this specification and the appended claims, singular forms such as “a,” “an,” and “the” may include the plural unless the context clearly dictates otherwise. Thus, for example, reference to “a bracket” may include an embodiment having one or more of such brackets, and reference to “the target plate” may include reference to one or more of such target plates.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing nearly all the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “generally” refers to something that has characteristics of a quality without necessarily being exactly that quality. For example, a structure said to be generally vertical would be at least as vertical as horizontal, i.e., would extend 45 degrees or greater from horizontal. Likewise, something said to be generally circular may be rounded like an oval but need not have a consistent diameter in every direction.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

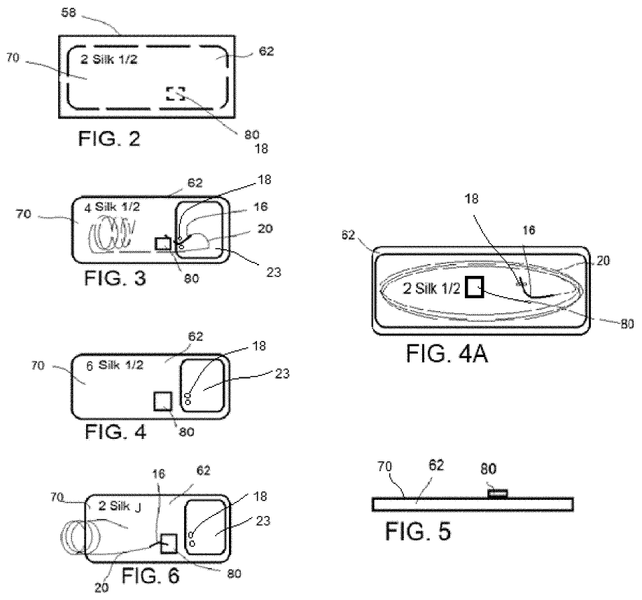

Turning now to FIG. 2, there is shown a surgical suture pack 58. The surgical suture pack 58 contains a suture tray 62, which may be a piece of molded plastic or cardboard. Disposed on the suture tray 62, or on a cover thereof, is a suture needle holding pad 80. The suture tray 62 and the suture needle holding pad 80 remain sterile within the surgical suture pack 58. When ready to use, the surgical suture pack 58 is removed.

FIG. 3 shows a surgical suture tray 62 (similar to that shown in FIG. 2, but for a different size thread) removed from the packaging. The surgical suture tray 62 holds a needle 16 and a suture thread 20. The needle 16 is held in a needle holder 18 by press fitting the needle between two plastic posts. Other types of needle holders also exist.

The suture tray may include an upper cover 70, typically formed by paper, cardboard or mylar coated material, which protects most of the suture material and the tip of the needle. The cover 70 may have information associated with the needle and/or the suture thread disposed thereon, such as the type of needle and the size and material of the suture material. Thus, the suture thread is shown in dashed line. The needle 16 is often held in the needle holder 18 on the bottom of the suture tray 62 so that the sharp end of the needle is covered by the upper cover 70 and is thus shown partially dashed. Other brand suture trays can be configured differently. The upper cover 70 may include an opening 23, through which the surgical technician or the physician can access the needle to remove it from the needle holder 18 and pull the suture material out through the opening.

The suture needle holding pad 80 may be disposed on the surgical suture tray 62 provided that a label having the needle/suture information is disposed thereon—thus allowing the cover 70 to be torn away as shown in FIG. 4A. (It will be appreciated in some current brands the suture tray is a small plastic tray and other brands use a folded piece of cardboard to form the tray. The present disclosure is intended to include both these and a variety of other trays which may be used to carry a suture needle and suture thread.) Preferably, the needle holder 18 and the needle holding pad 80 are spaced apart from each other so it is apparent when the needle has been removed from the needle holder and placed in the needle holder pad. For example, as shown in FIG. 3, the needle 16 is held in the needle holder 18 and not the needle holding pad which is on top of the cover. Thus, a surgical technician can readily tell that the needle has not yet been used.

FIG. 4 shows the surgical suture tray 62 with the needle 16 and the suture thread 20 removed. Such would occur when the surgeon is using the suture needle 16 and suture thread 20 to suture a patient. When the suture is completed, the surgeon would hand the suture needle and suture thread back to the surgical technician, who would when secure the suture needle 16 into the suture needle holding pad 80 on the tray, or the surgeon may simply directly mount the needle 16 into the needle holding pad 80 as shown in FIG. 6. Because the surgeon already has the needle gripped for use on the patient, inserting the sharp end of the needle into the needle holding pad 80 is extremely easy and efficient. If the surgeon were to try and remount the needle in the needle holder 18, it would likely take more time and be considerably more difficult, as the needle would have to be properly aligned between the posts and then press fit into place.

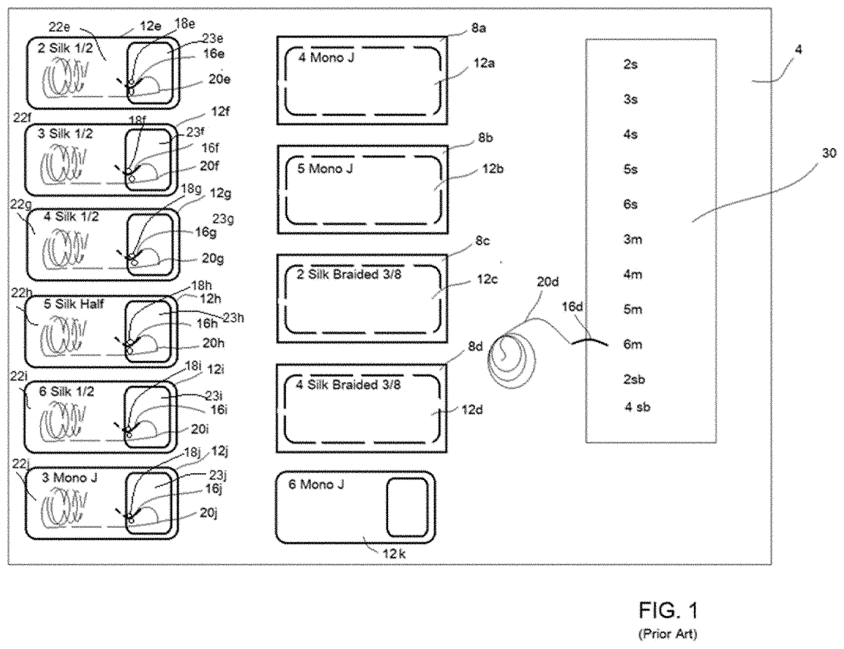

FIGS. 2, 3, 4 and 6 provide a small demonstration of the use of the present invention. The four suture trays 62 are presented to the surgical technician. He or she can look at the trays 62 and instantly deduce that, (1) the 2 Silk ½ has not been opened and is still sterile; (2) the 4 Silk ½ has been opened, but has not yet been used; (3) the 6 Silk ½ is currently being used; and (4) the 2 Silk J has already been used and returned to its packaging. Because each of the suture needles are attached to the suture tray 62 from which they are taken (other than the one which is in use), the surgical technician can easily track the location of each suture needle and suture thread based on the information which is disposed on the suture tray. There is no need to use a separate sponge or for the surgical technician to try and write down which needle/sutures have been used during the surgery. Moreover, in most situations, only one or at most two of the suture trays would be empty at any given time. Thus, the risk that a surgical technician might mistake one suture for another is significantly reduced. If a suture is missing, the surgical technician instantly knows which one is missing and a search based on when that suture was used can be undertaken. If the surgeon asks for a needle and there is an empty tray (other than the situation where it is known that two needles are being used contemporaneously), the surgical technician knows instantly that a needle has gone missing and needs to be located immediately. If the surgical technician had written the numbers in advance as shown in FIG. 1, this would not be possible.

It will be appreciated that in many surgical environments the number of suture trays 62 will be significantly higher. It is not uncommon to have 10-20 different suture trays in the operating field, and some procedures will have as many as 30 needle/suture combinations on a Mayo tray. The surgical technician is responsible for tracking the location of every one of the needle/suture combinations, as well as helping the surgeon by holding various instruments, etc. during the surgery.

FIG. 4A shows an embodiment where the bottom of the suture tray is provided with a label identifying the needle/suture combination, and a needle holding pad 80 disposed on the label and spaced apart from the needle holder 18 to ensure that the technician can readily determine if the needle has been used or not.

FIG. 5 shows a side view of the surgical suture tray 62 and the suture needle holding pad 80 disposed on top of the cover. It will be appreciated that the suture needle holding pad 80 can be any shape and need only be thick enough to receive and hold the needle. It can be made from a variety of materials including foam, rubber, neoprene, fabric. magnets or any other sterilizable material which will receive and temporarily hold the needle without damaging the needle.

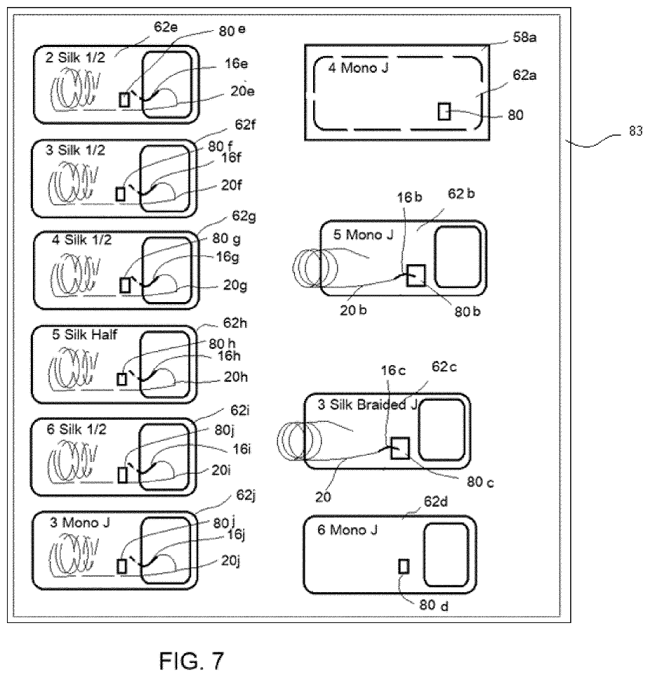

Turning now to FIG. 7, there is shown a top view of a surgical tray 83, often called a Mayo tray, having a suture package 58a and suture trays 62b-62j made in accordance with the present disclosure. The surgical technician can readily determine which surgical trays have been opened and which suture packages are not yet opened. The surgical trays can even be arranged in a manner which makes the procedure easiest for the surgical technician. For example, the trays can be arranged by which are open and which are not. Or the trays can be sorted by needle shape or suture material and size. By glancing at the suture trays 62b-62j, the surgical technician can tell that needles 16e through 16j have not yet been used. Needles 16b and 16c have been used and then secured in the suture needle holding pads 80b and 80c. Suture needle 16d (not shown) is currently in use and will be attached to the suture needle holding pad 80d when the surgeon returns it to the surgical technician. The tray 62d for the currently used needle could even be slid forward on the tray so that the surgeon can access it and mount the needle himself or herself without having to hand off the needle to the surgical technician. If a suture thread brakes, the needle can be attached to the associated suture needle holding pad and the surgical technician can see that the thread is broken. A second package of the same size and type may be opened and used without confusion at the sponge. Such an arrangement is far easier on the surgical technician and reduces work both before, during and at the end of the surgical procedure.

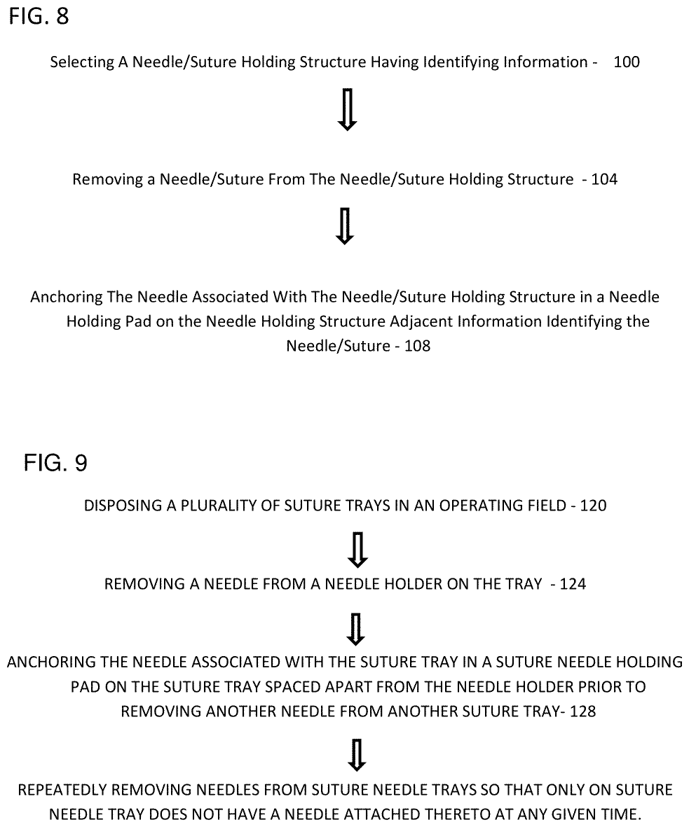

FIG. 8 shows a flow chart of steps which may be used in using and securing a surgical suture. The method involves selecting a suture tray 100. This may involve opening the packaging for a tray or selecting a tray which has been previously opened. As discussed below it can also include opening the envelope for a wet needle/suture. Thus, FIG. 8 refers to a needle/suture holding structure rather than just a tray.

The second step is removing the needle from the suture tray 104. This will typically involve removing the suture thread as well unless the suture thread breaks. The third step is anchoring the needle in the suture needle holding pad 108 when not in use by the physician. At appropriate times during the surgery the surgical technician can verify which needles have been removed but are not on their associated suture needle holding pads and can verify with the surgeon that he or she is using any needle that has been removed but is not on the pad. The method may include additional steps, such as relocating the needle/suture holding structures based on the design of the needle or size or material of the suture. The method may also include relocating the needle/suture holding structure based on whether or not it has been used, and or positioning a needle/suture holding structure to a position where it is more easily reached by the surgeon, or where the surgical technician can easily attach the needle to the needle holding pad.

FIG. 9 shows the steps of another method according to the present disclosure. This includes disposing a plurality of suture trays in an operating field, removing a needle from a needle holder on a tray and anchoring the needle associated with the tray in a needle holding pad on the suture tray apart from the needle holder prior to removing a needle from another suture tray.

The method may also include additional steps, such as relocating the suture trays based on whether they have been used, relocating the suture trays based on the needle and/or suture type or size and relocating the tray for the needle being used to make it more accessible.

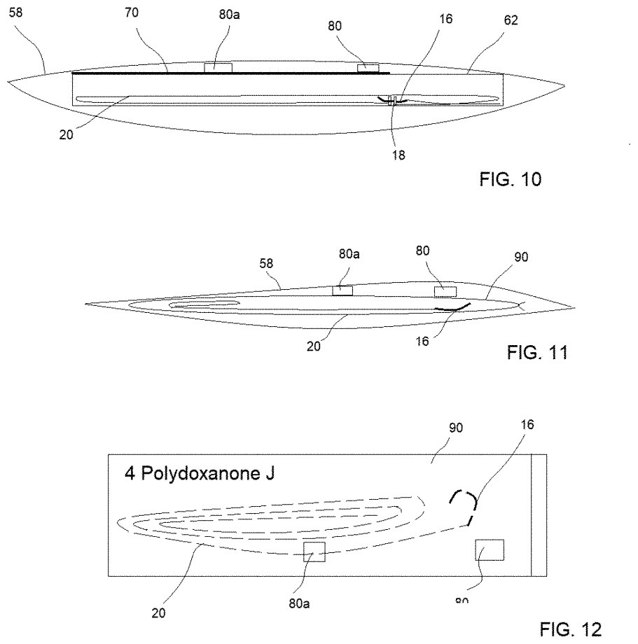

FIG. 10 shows a side view of a suture tray 62 disposed inside of packaging 58. The suture tray 62 includes a cover 70 which, though not visible in FIG. 10, has writing disposed thereon to identify the needle and suture material contained in the suture tray. Disposed on the cover 70 is a needle holding pad 80. Disposed in the suture tray 62 is a needle holder 18 for holding the needle 16 which is attached to the suture material 20. The cover 70 is typically formed with an opening 23 through which the needle 16 is grasped with forceps to remove the needle and suture material 20 from the suture tray 62. The needle holding pad 80 is placed a sufficient distance so that it can be clearly determined whether the needle is in the needle holder 18 or in the needle holding pad. This may be by placing it on a separate level—as shown in FIG. 10, and/or by moving the pad away from the needle holder as shown in FIG. 4A, or at needle holding pad 80a in FIG. 10.

While the previous embodiments show only a single needle holding pad, it will be appreciated that the multiple needle holding pads could be located on the cover 70, etc. with the different locations providing information to be remembered by the surgical technician. For example, if the surgeon thinks that he or she has used enough of the suture material that there is an insufficient amount left on that needle, or the needle has been damaged by striking bone, has become contaminated, etc., the surgeon may instruct the surgical technician to not reuse that needle. The needle could then be placed on needle holding pad 80a indicating that it is not to be reused, while the needle/suture combinations for reuse are mounted on needle holding pad 80 indicating that they are available for reuse. Because of the packaging material 58, the needle holding pads 80, 80a remain sterile prior to use.

FIG. 11 shows a side view of alternate packaging for needle/suture combinations. Some suture material must remain moist prior to use in order to function property. To maintain moisture, the needle 16 and suture material 20 is contained inside a waterproof envelope 90 prior to use. The waterproof envelope usually contains information about the needle and suture material similar to the covers 70 discussed above. The waterproof envelope 90 is usually contained in a protective packaging 58 which likewise contains information about the needle and suture.

In accordance with one aspect of the present disclosure, a needle holding pad 80 is attached on the outside of the waterproof envelope 90. In use, the packaging 58 is removed and then the envelope 90 is torn or otherwise opened and the needle and suture material removed. The needle 16 and suture material 20 are then used. When finished, the needle 16 can be mounted in the needle holding pad 80 to show that the needle has been used and that it is available for reuse. If a second needle holding pad 80a is included, the holding pads could be color coded to indicate whether the needle is reusable or not.

FIG. 12 shows a top view of the envelope 90 of FIG. 11. A first needle holding pad 80 and a second needle holding pad 80a are disposed on the outside of the envelope so that after the envelope has been opened and the needle 16 and suture material 20 removed, the needle can be secured in the needle holding pad 80 or 80a.

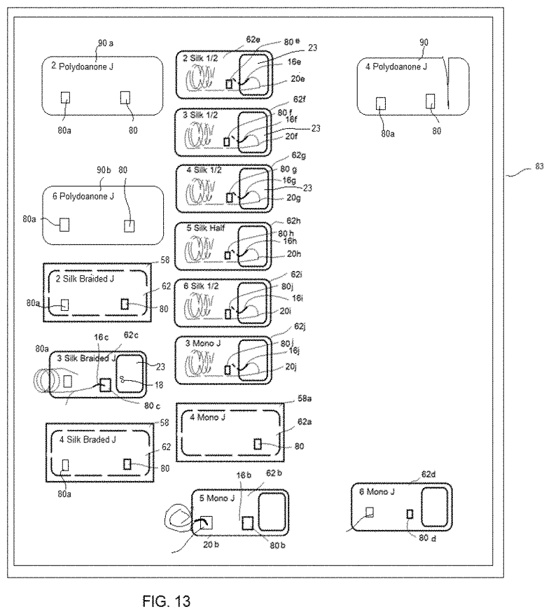

FIG. 13 shows a top view of a Mayo tray 83 with a plurality of needle/suture trays and envelopes to demonstrate one example of needle/suture management in accordance with the present disclosure. Beginning on the left side, there is shown two needle suture envelopes 90a and 90b which each hold a needle and a wet suture as discussed above with respect to FIGS. 11 and 12. The two envelopes are disposed in order based on the size, with a gap positioned between them because the needle/suture from the 4 Polydoxanone with a J needle is currently in use and the envelope for that needle/suture combination has been moved to the side of the Mayo tray 83 closest to the surgery.

Specifically, the right-hand side of the Mayo tray may represent a situation where two surgeons are suturing at the same time. The envelope 90 is disposed in the upper corner, consistent with a surgeon who is to the left of the surgical technician, who would be to the left of the Mayo tray 83. The surgical technician thus knows that surgeon 1 to the left is using the needle/suture from envelope 90. On the bottom of the tray in FIG. 13, which would be to the right of the surgical technician, the tray 62d for the size 6 Mono with a J hook is in use by surgeon 2. A surgeon may be able to reach the tray/envelope associated with their needle/suture combination and simply insert the needle into the needle holding pad 80, 80d. Alternatively, the surgical technician can receive the forceps and mount the needle. This could even be done if both surgeons hand back their forceps at the same time and the risk that the surgical technician will confuse the needle/suture combinations is virtually eliminated.

The various envelops 90-90b and trays 62-62e can be arranged in any arrangement that works for the surgical technician. They can be sorted by needle, by suture material, by which ones have been used, which are open and which are still in the packaging. As shown on tray 62b, they can even be sorted to include those where the needle is no longer useable (signified by the needle being disposed in needle holding pad 80a). Both the surgical technician and the surgeons can look to verify the status of their needle/suture combinations and ensure that all needles are accounted for.

Thus, there is disclosed a method and apparatus for holding suture needles. It will be appreciated that modifications can be made without departing from the scope and spirit of the invention. The appended claims are intended to cover such modifications.

What is claimed is:

1. A method for securing needles and suture material during a surgical procedure, the method comprising:
selecting a container having an exterior surface and having a needle holding pad disposed thereon and a needle and suture material attached to the needle disposed within the container;
removing the needle and suture material from the container and
attaching the needle to the needle holding pad on the exterior surface of the container.

2. The method according to claim 1, wherein the container comprises an envelope having writing identifying the type of needle and suture materials disposed therein and wherein the needle holding pad is disposed adjacent to the writing.

3. The method according to claim 1, wherein the method includes mounting the needle in the needle holding pad after the needle has been used to perform a surgical suture.

4. The method according to claim 1, wherein the method comprises using a plurality of containers and attaching each needle to the container from which it was removed.

5. The method according to claim 4, wherein the method comprises arranging the containers by type of needle and suture material which the container contained.

6. The method according to claim 5, wherein the method comprises rearranging the containers as they are used.

7. The method according to claim 1, wherein the method comprises isolating a container which has had the needle and suture material removed, but has not yet had the needle attached to the needle holding pad.

8. The method according to claim 1, wherein the method comprises attaching a needle to the needle holding pad so as to indicate that the needle is not to be reused.

9. A method for securing suture needles, the method comprising:
selecting a suture tray having a cover and a suture needle disposed within the tray under the cover and a needle holding pad disposed on top of the cover;
removing the needle from the suture tray; and
inserting the needle into a suture needle holding pad disposed on top of the attached to the suture tray.

10. The method according to claim 9, wherein the suture tray has information correlated to the needle and/or to the suture thread disposed on the suture tray.

11. The method according to claim 9, wherein the method comprises selecting a suture tray having a suture needle holding pad disposed on a top side thereof.

12. The method according to claim 10, wherein the method comprises setting the suture tray on a surgical tray having a plurality of suture trays disposed thereon.

13. A surgical suture container comprising:
A container having a needle holding pad disposed on an exterior surface of the container and a needle and suture material disposed on the interior of the container.

14. The surgical needle container of claim 13, wherein the container is a sealed envelope containing a moistened suture material and wherein the needle holding pad is disposed on the top of the envelope.

15. The surgical needle container of claim 14 wherein the envelope is waterproof and wherein the needle and suture material are removed from the container by ripping the envelope.

16. The surgical needle container of claim 13, wherein the envelope has writing disposed thereon identifying the type of needle and suture material disposed in the envelope.

17. The surgical suture tray according to claim 14, wherein the container comprises a suture tray which has an upper surface and wherein the suture needle holding pad is disposed thereon.

18. The surgical suture tray according to claim 14, wherein the suture tray has a cover and wherein the suture needle holding pad is disposed on the cover.

19. The surgical suture tray according to claim 17, wherein the cover includes information disposed thereon identifying at least one of a needle size, a needle shape, and a suture size.

20. The surgical suture tray according to claim 19, wherein the suture tray has a needle disposed below the cover.
